(19) United States
(12) Patent Application Publication
(10) Pub. No.: US 2002/0072932 A1
(43) Pub. Date: Jun. 13, 2002

(54) Health personal digital assistant

(76) Inventor: Bala Swamy, Danville, CA (US)

Correspondence Address: William M. Hanlon, Jr., Young & Basile, P.C., 3001 West Big Beaver Road, Suite 624, Troy, MI, 48084, US

(21) Appl. No.: 09/734,859

(22) Filed: Dec. 11, 2000

(51) Int. Cl. G06F 17/60; A61B 5/00; A61B 5/08

(52) U.S. Cl. 705/2; 600/300; 600/531

(57) ABSTRACT

The present invention is a personal health management device comprising a processor executing an operating program, input means and output means. The input means receives information about an individual through various sources, including nutritional information about food ingested, biological information, and the caloric expenditure of the individual's activities. Preferably, input from the various sources occurs in real-time through wireless communications means. Input can also be obtained from internet websites and from health care providers, such as doctors. The operating program uses these inputs to output a health report using the output means, preferably on a display. The report can also be provided to health care providers. In one aspect of the invention, the output is a signal capable of operating a pharmaceutical delivery device carried on the individual. The personal health management device of the present invention provides a convenient means for a user to monitor his health.

### BACKGROUND OF THE INVENTION

1. Field of the Invention

The present invention relates, in general, to personal health software based systems.

2. Description of the Art

More people are trying to monitor and evaluate their health, both for medical and personal reasons. As part of this effort, many have developed personal health programs monitoring both exercise and diet. Others, such as those with diabetes, must additionally perform tests to monitor specific physiological parameters. Individuals must then compare the data with input from such resources as physicians and other experts to reach a reasonable conclusion regarding existing physical condition.

Traditionally, monitoring exercise and diet has involved a great deal of data. To assess caloric intake, the individual must document the amount and type of food eaten, go through tables to look up the caloric content of items, and manually track and record totals. To assess caloric output—calories expended—an individual must determine his metabolic rate for each activity undertaken, consult long lists of exercises to determine the amount of calories burned for each activity based on metabolic rate, and manually track and record totals.

In addition to exercise and diet, many individuals must periodically measure certain physiological parameters. For example, diabetics must measure blood glucose concentration, often several times a day. Similarly, the measurement of blood cholesterol concentration provides important information on coronary artery disease. Once the magnitude of a particular parameter is reported, often the individual must compare it to an acceptable level and take pro-active measures.

Finally, physicians have traditionally supplied base information that individuals use for comparison, such as ideal weight and acceptable levels of blood glucose. Information received from other sources, such as Internet health-related sites, far exceed the information provided only by doctors.

The wealth of resource information available, and the amount of information that must be recorded to make a meaningful health assessment, has grown exponentially as scientific knowledge has progressed. For example, mere measurement of caloric content in food is no longer sufficient to assess its affects on human health. Such parameters as fat and sugar content are also important. This information overload has proven to be an all but insurmountable barrier to many individuals, even those considered health-conscious.

Technology has provided a partial response to this challenge. For example, the personal computer has helped to monitor exercise and diet. Software programs now exist that establish target weights and daily diet and exercise plans using extensive food and exercise information pre-programmed into the computer's memory. These programs, however, still require that the user document exercise and diet for later manual input. Moreover, they do not generally accept input of real-time biological parameters received through self-testing. Nor do they alarm and/or control a pharmaceutical delivery system.

Lack of mobility makes desktop computers impracticable for monitoring real-time health. Development of new computers has focused on miniaturization in an effort to support user mobility. In the last few years, this effort has led to the development of the personal digital assistant (PDA). PDAs are light weight, hand-held computers designed to run such applications as word processors, spreadsheets, and calendars and address books. Moreover, PDAs have communications capabilities, typically wireless, for sending and receiving data and messages. A PDA can also be synchronized and backed up to a desktop computer.

Thus, it would be desirable to develop a software based system capable of receiving various inputs using the wireless communications capability of a PDA, analyzing the inputs to assess user health, and reporting various outputs, including a detailed health report and recommendations. It would also be desirable to include an output signal that would report the need to take a medication and/or control dispensing of the medication through a pharmaceutical delivery system.

### SUMMARY OF THE INVENTION

The present invention is a software based system taking advantage of the wireless communications capabilities and easy transportability of a PDA to receive various inputs in real-time or near real-time and produce immediately responsive output related to an individual user's health. The invention receives as inputs various nutritional, biological and exercise related information and sends as output a customized health report and, optionally, a signal to a pharmaceutical delivery system.

Specifically, the invention is a personal health management device, comprising a processor executing an operating program; input means, coupled to the processor, for receiving and inputting to the processor at least one of food sample nutritional information, biological information and activity caloric expenditure information of a user; and output means coupled to the processor. The processor is responsive to the input means and executes the operating program to generate a health report and the output means outputs the health report.

The input means is responsive to at least one of an exercise device transmitting means external of the processor, for providing activity caloric expenditure information of a user using the exercise device; a real-time oxygen measuring device transmitting means external of the processor, for providing activity caloric expenditure information of the user; a food sample nutritional information measuring device transmitting means external of the processor, for providing food sample nutritional information of a food sample; and a biological information measuring device transmitting means external of the processor, for providing biological information of the user.

The input means of the invention can further include communication means. In one aspect of the invention, the communications means can communicate with a global telecommunications network. In another aspect, the communications means includes wireless communication means for communicating with at least one external device. For example, the input of information from an exercise device can occur via the wireless communications means.

In one aspect of the invention, the output means comprises a display that outputs the health report. In another aspect of the invention, the output means further comprises means responsive to a procedure for generating activation signals adapted to control a pharmaceutical delivery system carried on the user.

In a final aspect of the invention, the processor, the input means and the output means are disposed in a handheld housing. If the invention includes a memory, the memory is also disposed in a handheld housing.

### BRIEF DESCRIPTION OF THE DRAWINGS

The various features, advantages, and other uses of the present invention will become more apparent by referring to the following detailed description and drawing in which:

FIG. 1 is a general block diagram of the various inputs used by and outputs generated by the present invention;

FIG. 2 is a simplified diagram of the hardware architecture of the personal digital assistant (PDA) of the present invention shown in FIG. 1;

FIGS. 3A and 3B are block diagrams illustrating two different methods of calculating nutritional information of food as inputs into the present invention;

FIG. 4 is block diagram demonstrating a possible method of creating a database of chemicals/nutrients used in calculating the nutritional information of food;

FIG. 5 is a flow diagram showing the various biological information used as inputs into the present invention;

FIG. 6 is a block diagram showing how the system of the present invention uses biological information to generate an output regarding the need for pharmaceutical delivery;

FIG. 7 is a flow diagram demonstrating the various means of gathering a user's caloric output as an input into the present invention; and

FIG. 8 is a block diagram showing how the system of the present invention uses the various inputs to generate one potential version of a health report; and

FIG. 9 is a block diagram showing how the dosage information needed to properly signal the pharmaceutical delivery system is input into the system of the present invention.

### DETAILED DESCRIPTION

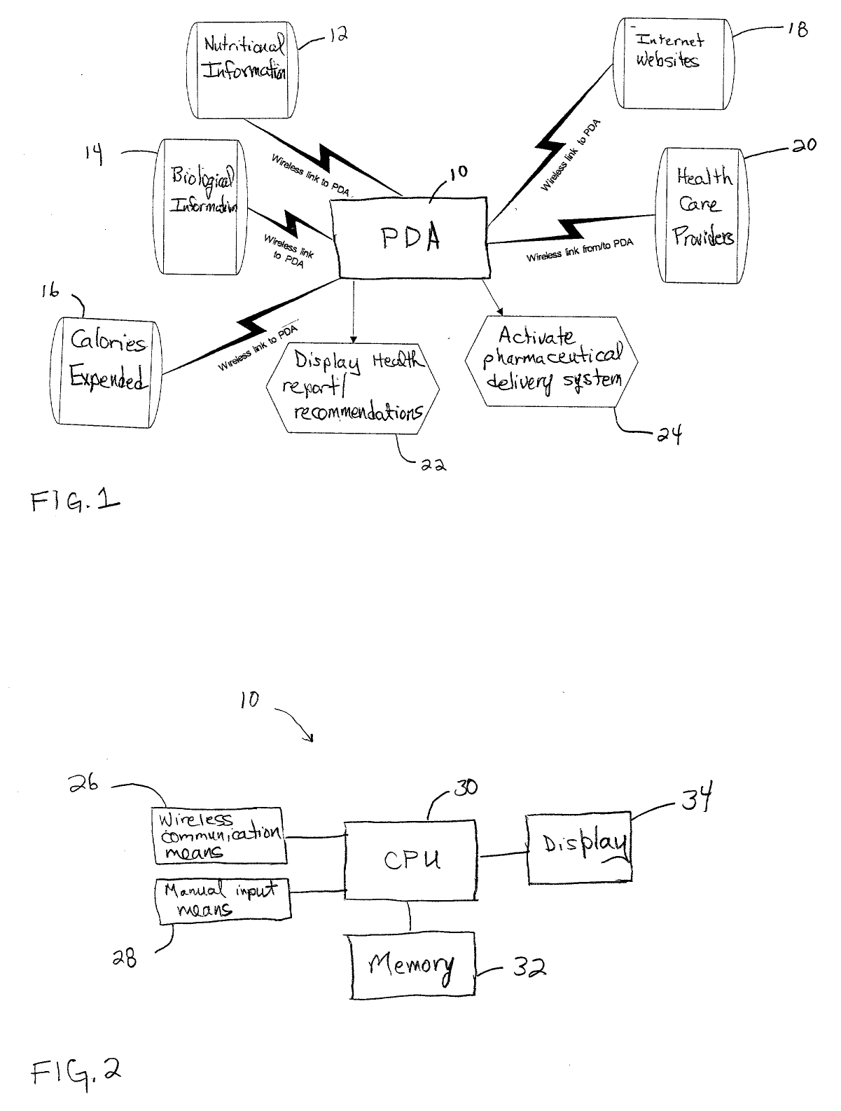

Referring to FIG. 1, there is depicted a diagram of the inputs and outputs of a health management software system according to the present invention. The inventive system includes use of a PDA 10. As shown in FIG. 2, the PDA 10 is of conventional construction, comprising wireless communication means 26 (hereinafter wireless links) capable of both receiving and transmitting data, a manual input means 28, either stylus or keyboard, a central processing unit 30 (CPU) with memory 32, and a display 34. Typical PDAs are sold by Palm, Psion and Visor, to name a few.

Referring back to FIG. 1, the system in the PDA 10 receives data from wireless links 26 to input data, such as nutritional information about food 12, biological information 14, and calories expended during daily activities 16. Additionally, wireless links 26 to Internet websites 18 and health care providers 20, such as doctors and insurance companies, supply input on health goals and needs. The system sends as output to the PDA 10 a personal health report 22 to the user, which can include, for example, an assessment of health goals and recommendations of exercise and diet. The report 22 is produced each time an input changes, or upon a user's prompt. Optionally, this report 22 could be furnished directly to health care providers 20. In one aspect of the present invention, the system sends as part of the health report 22 a message that medications are needed. In another aspect, the system signals to activate a pharmaceutical (drug) delivery system 24 through a wireless link 26.

The nutritional information 12 regarding food consumed by the user is preferably calculated using techniques including, for example: x-ray holography, ultrasonics, spectrography, such as Raman or nuclear magnetic resonance (NMR) spectroscopy, or calorimetry. Spectroscopy, for example, has already been used in non-invasive methods of measuring biological substances, as described in U.S. Pat. Nos. 5,553,616; 5,243,983; and 5,685,300, which patents are incorporated herein by reference. Ultrasonic techniques have also been used widely in biomedical applications. Raman spectroscopy is the preferred technique.

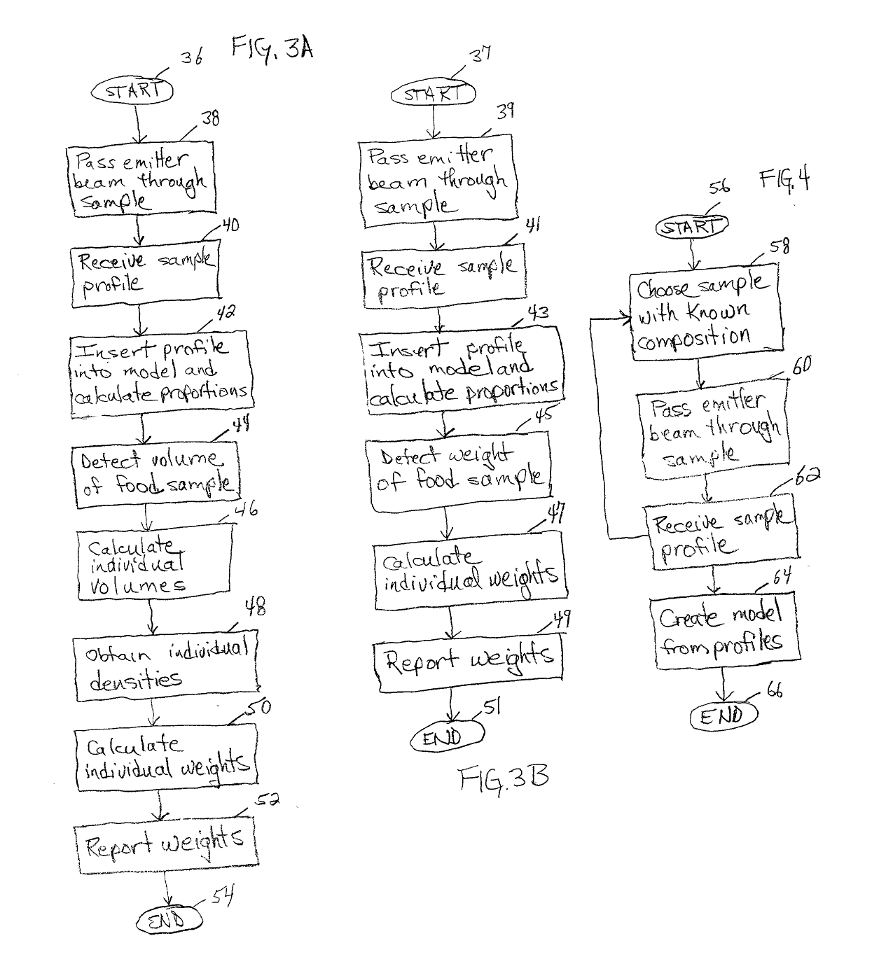

Referring now to FIGS. 3A and 3B, illustrated are possible procedures by which the aforementioned techniques are used to input nutritional information 12 about food into the system of the present invention. Generally, this involves two main stages: (1) inserting a profile (ultrasonic, spectroscopic, or otherwise) obtained from a food sample into a model developed through profiles of known compositions to determine the proportion of each chemical/nutrient in the sample; and (2) calculating the weight (the nutritional information 12) of each chemical/nutrient in the sample by measuring the sample. Whether the procedure of FIG. 3A or FIG. 3B is followed depends upon whether the model is developed using a samples gathered by weight or by volume, but initially the procedures are the same.

Referring now to FIG. 3A, the procedure begins with the first stage, a determination of the proportion of chemicals/nutrients in a sample, in step 36. It proceeds to step 38, where the food sample is scanned by passing a beam from an emitter through the food sample, then to step 40, where the profile of the reflected beam is detected by a receiver. The emitter could supply a beam from a low-powered laser source or a magnetic field source. Preferably, the emitter and receiver used in steps 38 and 40 are hardware incorporated into the capabilities of the PDA 10, emitting and receiving signals through the wireless links 26. In step 42, the resulting profile is inserted into a model to predict the proportion of each chemical/nutrient in the food sample. The results of this prediction step would be proportions of each chemical/nutrient detected as a percentage by volume of the total food sample.

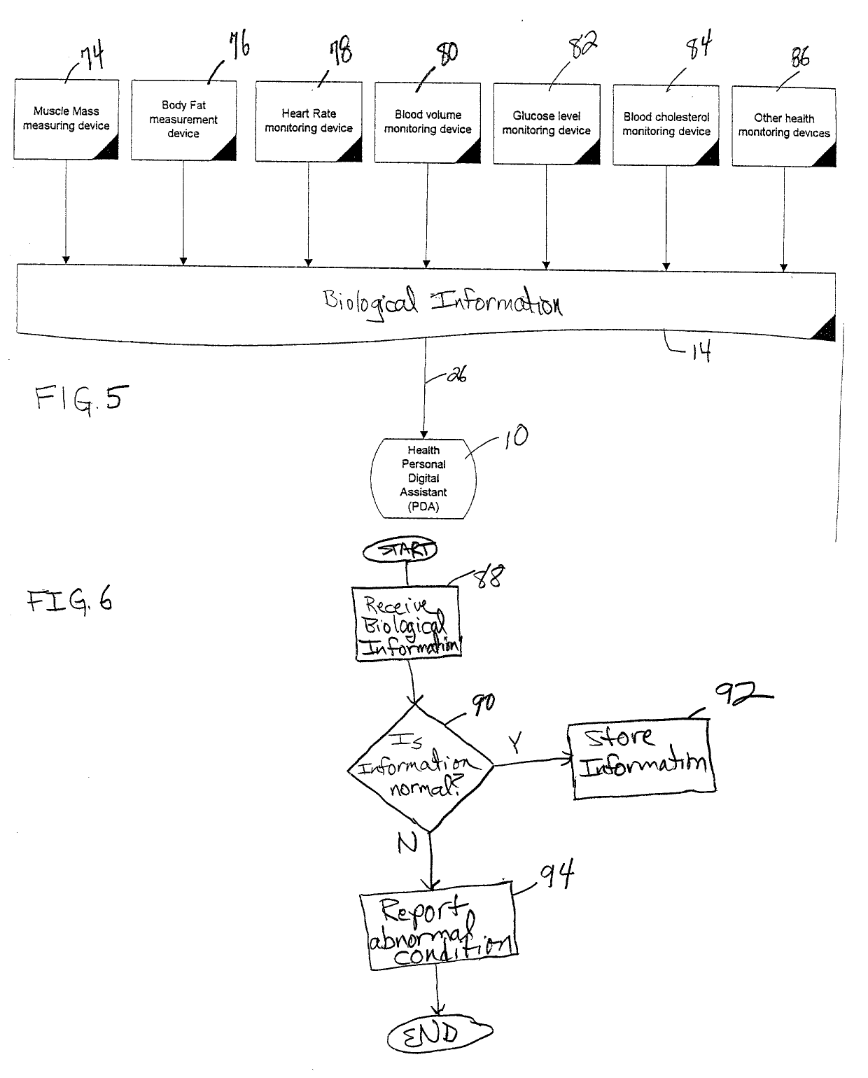

One method of creating this model is illustrated in FIG. 4, starting with step 56. In step 58, a calibrating sample with a known composition is chosen, i.e., the volume or weight or both of each chemical/nutrient in the calibrating sample is known. For example, the fat could be 50% and carbohydrates could be 50%. Then, it is scanned by passing at least one beam from an emitter through the calibrating sample in step 60, and the profile of the reflected beam is detected by a receiver in step 62 and stored. The emitter could supply a beam from a low-powered laser source or a magnetic field source. These steps are then repeated beginning at step 58 for a new calibrating sample of known composition until a statistically significant sample size for each chemical/nutrient is analyzed. Then, in step 64, the stored profiles are used to build, optimize and test a model. The model would predict the proportions of each chemical/nutrient in an input profile as a percentage by volume or a percentage by weight or both and could be created using a variety of chemometric software programs. Some vendors of chemometric software programs include Infometrix, Inc. of Woodinville, Washington and Applied Chemometrics of Sharon, Mass. Preferably, this model is stored in the memory 32 of the PDA 10. The creation of the model ends at step 66.

Returning now to FIG. 3A, after the proportion of each chemical/nutrient in the food sample is determined in step 42, it is used in the second stage to determine the weight (the nutritional information 12) for each chemical/nutrient in the sample. Determining the weight of each chemical/nutrient begins at step 44, where the volume of the food sample is measured with a volumetric sensor. The volumetric scanner could be any one of a variety of scanners that uses different techniques to determine volume. One scanner is an image scanner, where the scanner determines the volume based on the profile of the food sample. These scanners are currently used in medical applications to determine the volume of an organ, for example, lungs. Another scanner is a molecular volumetric scanner, which scans for the total volume of all molecules in the sample. Regardless of the scanner used, it is preferred that the volumetric sensor is hardware incorporated into the PDA 10, using the wireless links 26 to send and receive data. After the total volume is measured in step 44, the volume of each chemical/nutrient identified in step 42 is calculated in step 46 according to the following formula:

volume of chemical/nutrient=percentage of chemical/nutrient (by volume)*volume of food sample.

By example, if the percentage by volume of chemicals/nutrients identified in step 42 include fat (10%), carbohydrates (20%), vitamin A (3%), sodium (4%) and cholesterol (30%), and the volume of food is 300 cc, then the volumes calculated in step 46 would be: 30 cc of fat, 60 cc of carbohydrates, nine cc of vitamin A, 12 cc of sodium, and 90 cc of cholesterol.

Once the volume of each chemical/nutrient is calculated in step 46, the density of each chemical/nutrient is obtained from a database of chemicals/nutrients and their densities in step 48 Preferably, this database would be stored in the memory 32 of the PDA 10. In step 50, the densities obtained in step 48 are used to calculate the weights of the individual chemicals/nutrients identified in step 42 according to the following formula:

weight of chemical/nutrient=volume of chemical/nutrient*density of chemical/nutrient.

For example, assuming the volumes calculated in step 46 above and densities of 0.667 g/cc for fat, 0.167 g/cc for carbohydrates, 0.222 g/cc for vitamin A, 0.5 g/cc for sodium, and 0.167 g/cc for cholesterol, the weights calculated in step 50 would be: 20 grams of fat, 10 grams of carbohydrates, two grams of vitamin A, six grams of sodium, and 15 grams of cholesterol. After reporting this nutritional information 12 to the system of the present invention in step 52, this procedure ends at step 54.

Referring now to FIG. 3B, shown is an alternative procedure for determining the nutritional information 12 for input into the present invention when the model described in FIG. 4 predicts chemicals/nutrients as a percentage by weight, not volume as in FIG. 3A. As in FIG. 3A, such a procedure begins with the first stage, a determination of the proportion of chemicals/nutrients in a sample, in step 37 of FIG. 3B. It proceeds to step 39, where the food sample is scanned by passing a beam from an emitter through the food sample, then to step 41, where the profile of the reflected beam is detected by a receiver. Again, the emitter could supply a beam from a low-powered laser source or a magnetic field source. Preferably, the emitter and receiver used in steps 39 and 41 are hardware incorporated into the capabilities of the PDA 10, emitting and receiving signals through the wireless links 26. In step 43, the resulting profile is inserted into a model to predict the proportion of each chemical/nutrient in the food sample. The results of this prediction step would be proportions of each chemical/nutrient detected as a percentage by weight of the total food sample.

In step 45, the total weight of the food sample is detected using a weight scanner. The weight scanner could be any one of a variety of scanners that uses different techniques to determine weight. One scanner, for example, is a molecular weight scanner, which scans for the total weight of all molecules in the sample. Regardless of the scanner used, it is preferred that the weight sensor is hardware incorporated into the PDA 10, using the wireless links 26 to send and receive data. After the total weight is measured in step 45, the weight of each chemical/nutrient identified in step 43 is calculated in step 47 according to the following formula:

weight of chemical/nutrient=percentage of chemical/nutrient (by weight)*weight of food sample.

By example, if the percentage by weight of chemicals/nutrients identified in step 43 include fat (20%), carbohydrates (10%), vitamin A (2%), sodium (6%) and cholesterol (15%), and the weight of food is 100 grams, then the weights calculated in step 47 would be: 20 grams of fat, 10 grams of carbohydrates, two grams of vitamin A, six grams of sodium, and 15 grams of cholesterol. After reporting this nutritional information 12 to the system of the present invention in step 49, this procedure ends at step 51.

As mentioned, the preferred method of inputting nutritional information 12 into the system of the present invention is through direct measurement techniques wherein the emitter, receiver, and sensor used in the measurements are incorporated as hardware into the PDA 10, and each model and database, if required, used to create the nutritional information 12 from these measurements is stored in the memory 32 of the PDA 10. Alternately, a stand alone device could use one of the specified techniques to calculate the nutritional information 12 using databases stored in its memory and transmit the results to the PDA 10 through a wireless link 26. Less preferred is indirect measurement, where the PDA 10 receives input from an external device designed to accept manual inputs of food consumed and to calculate nutritional information 12 from that input. For example, U.S. Pat. No. 5,890,128, which is incorporated herein by reference, discloses a hand held device that accepts manual inputs of food items consumed and calculates caloric and fat content.

FIG. 5 illustrates possible biological information available as inputs into the software system of the present invention. Existing health monitoring devices are used to develop inputs transmitted to the PDA 10, preferably by means of wireless links 26. The possible devices are those that measure: muscle mass 74; body fat 76; heart rate 78; blood volume 80; glucose level 82; blood cholesterol 84; and other devices 86 such as devices that measure weight and height. For example, U.S. Pat. No. 5,553,616 discloses a method and apparatus for determining concentrations of various biological substances. U.S. Pat. No. 5,243,983 discloses a method and apparatus for determining the concentration of a Raman active molecule, preferably glucose 82. U.S. Pat. No. 5,685,300 discloses a method of measuring the concentration of both glucose 82 and cholesterol 84. A method and system to measure muscle mass 74 or body fat 76 is disclosed in U.S. Pat. No. 5,941,825, which is incorporated herein by reference. Real-time systems used to measure biological substances are not commercially available. However, for the measurement of glucose, for example, the systems closest to Food and Drug Administration approval are the GlucoWatch Biographer by Cygnus, Inc. of Redwood City, Calif. and the CGMS by MiniMed, Inc. of Sylmar, Calif. Preferably, the devices produce readings transmitted to the PDA 10 as inputs by means of wireless links 26. However, the manual input means 28 of the PDA 10 could also be used to input the information from these devices.

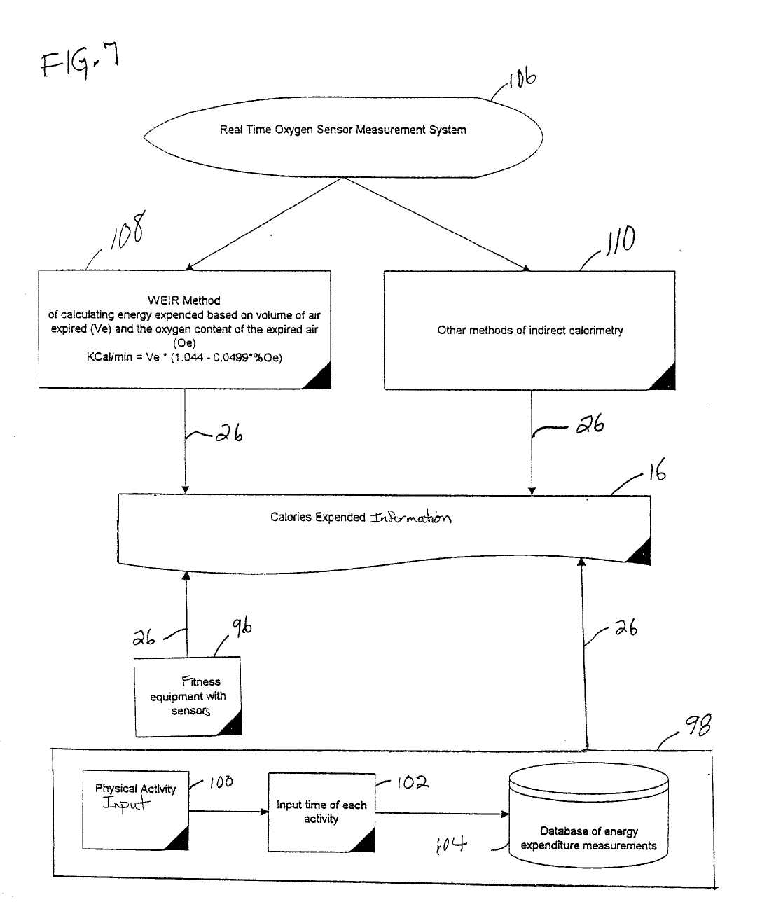

As one example of the use of the biological information 14, refer to FIG. 6. Once the biological information 14 is input into the PDA 10 in step 88, it is compared in step 89 to a database of normal conditions. The database is created using information input from health care providers 20. If all biological information 14 is normal, the biological information 14 is merely stored in step 90, and the procedure ends. If any of the biological information 14 is abnormal, then the system checks in step 91 whether it has the capability to signal the pharmaceutical delivery system 24. If the system does not, the PDA 10 reports the abnormal condition in step 92. Preferably, the abnormal condition is included in the health report 22. Alternatively, reporting an abnormal condition in step 92 involves the sounding of an alarm. The procedure then ends.

Returning to step 91, if the system can signal the pharmaceutical delivery system 24, the procedure checks dosage information in step 93. The dosage information is input into the system of the PDA 10 as shown in FIG. 9. Returning to FIG. 6, based on the dosage information received in step 93, the system then signals the delivery system 24 to deliver the correct pharmaceutical in step 94. Such a delivery system 24 could dispense vitamins or medications using a transdermal patch or a pump permanently lodged in the user's body. Pager-sized insulin pumps controlled by a computer chip designed to be worn 24 hours a day are already available through several manufacturers. The smallest currently available is the Disetronic Dahedi 25 from Disetronic Medical Systems USA, in Minneapolis, Minn. After the signal is sent in step 94, the procedure ends.

In FIG. 7, the various sources for calculating calories expended 16 by a user used as inputs into the software system of the present invention are shown. Preferably, the PDA 10 is capable of receiving calories expended 16 through wireless links 26 from existing sensor technology available with many exercise machines 96, including such devices as treadmills, pedometers and rowing machines, among others.

The PDA 10 is also capable of receiving calories expended 18 from a separate device 98, portable or otherwise, that calculates calories expended generally by using as inputs a user's exercise activities 100, the amount of time expended in the activities 102 and a database 104 of activities and their related caloric expenditures. Such a device is disclosed in U.S. Pat. No. 5,890,128. Preferably, the software system of the present invention receives this information from the separate device 98 through a wireless link 26. In an alternative aspect of the present invention, the PDA 10 incorporates this method of calculating calories expended 16, which is then used as an input into the software system of the present invention.

Finally, FIG. 7 shows that the PDA 10 is capable of receiving calories expended 16 by use of a real-time oxygen sensor measurement system 106. The oxygen sensor measurement system 106 would sense the real-time volume of air expired by the user and the oxygen content of the expired air and calculate the calories expended 16 using the WEIR method 108, or other methods of indirect calorimetry 110, such as closed-circuit and open-circuit spirometry. In the WEIR method, for example, the calories expended per minute are calculated using the volume of air expired by the user (Ve) and the oxygen content of the expired air (%Oe) in the following relationship:

calories expended per minute (kcal/min)=Ve*(1.044-0.0499* %Oe).

The user would indicate to the oxygen sensor measurement system 106 when to begin and end recording real-time expiration of air, then the calories expended would be calculated by multiplying the total time by the calories expended per minute, as calculated above. For further details on the WEIR system, see McArdle, et al., Essentials of Exercise Physiology, 2nd ed. (Lippincott, Williams and Wilkins 1999), which is incorporated herein by reference.

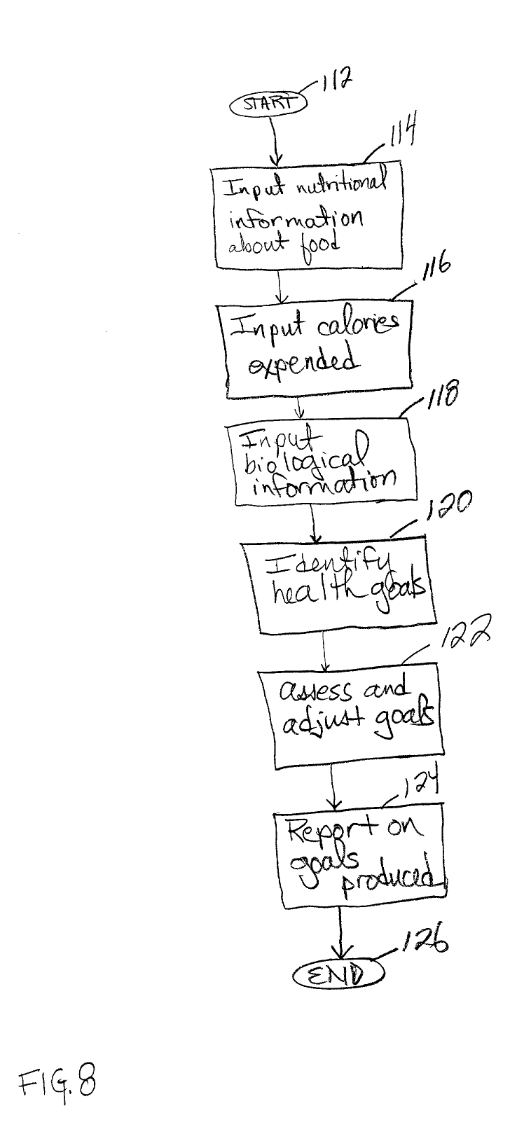

FIG. 8 shows how the system of the present invention uses the various inputs to generate a health report 22, including certain recommendations. The procedure starts with step 112, and proceeds to step 114, where nutritional information 12 about food is input. In step 116, calories expended 16 are input, and in step 118, biological information 14 is input. In step 120, health goals of the user are identified. These health goals could include weight loss, strength training or muscle toning, and can be input manually or from other external sources, such as internet websites 18 or health care providers 20. In step 122, the health goals identified in step 120 are assessed and adjusted based on the inputs. Then, in step 124, a health report 22 would be produced containing recommended training exercises and diet, including an assessment of the progress towards the user's goals. The particular contents of the health report 22 are by example only. As another example of the contents of the health report 22, the contents could merely summarize the inputs. The health report 22 could be produced upon prompting by a user, or could be produced each time an input changed. The procedure ends at step 126.

As previously mentioned, this health report 22 preferably includes the reporting of an abnormal condition In one aspect of the invention, the system would signal a pharmaceutical delivery system 24 in the event of an abnormal condition, as shown in step 94 of FIG. 6. Alternatively, the system could signal a delivery of pharmaceuticals according to a predetermined schedule of delivery. FIG. 9 shows how the system of the present invention would receive dosage information needed to signal the pharmaceutical delivery system 24. The procedure to gather this information for use by the PDA 10 in signaling the pharmaceutical delivery system 24 begins with step 128, and proceeds to step 130, where the PDA 10 receives insurance and history information, preferably through the wireless links 26, about the patient from health care providers 20. Alternatively, the information would be manually input through the manual input means 28. In step 132, whether a particular doctor authorized under an insurance plan is queried. If the answer is no, such information is reported in the health report 22 or otherwise in step 134. The procedure then ends at step 136.

Returning to step 132, if the particular doctor is authorized to see the patient, then the patient sees the doctor. Information on pharmaceuticals is then received from the doctor in step 138. Such information would include name of the pharmaceutical, its dosage amount, and information on when it should be dispensed. For some pharmaceuticals, dispensing information would be a dosage schedule comprising dates and times. For others, dispensing information would indicate which biological information 14 must be reported as abnormal for the particular pharmaceutical to be delivered upon a signal from the PDA 10. Once this pharmaceutical information is in the system of the PDA 10, the procedure ends at step 136.

What is claimed is:

1. A personal health management device, comprising:
a processor executing an operating program;
input means, coupled to the processor, for receiving and inputting to the processor at least one of food sample nutritional information, biological information and activity caloric expenditure information of a user;
the processor responsive to the input means and executing the operating program for generating a health report; and
output means, coupled to the processor, for outputting the health report.

2. The device of claim 1 wherein:
the input means is responsive to an exercise device transmitting means external of the processor, for providing activity caloric expenditure information of a user using an exercise device.

3. The device of claim 1 wherein:
the input means is responsive to a real-time oxygen measuring device transmitting means external of the processor, for providing activity caloric expenditure information of a user.

4. The device of claim 1 wherein:
the input means is responsive to a food sample nutritional information measuring device transmitting means external of the processor, for providing food sample nutritional information of a food sample.

5. The device of claim 1 wherein:
the input means is responsive to a biological information measuring device transmitting means external of the processor, for providing biological information of a user.

6. The device of claim 1 wherein the input means is responsive to at least one of:
an exercise device transmitting means external of the processor, for providing activity caloric expenditure information of a user using an exercise device;
a real-time oxygen measuring device transmitting means external of the processor, for providing activity caloric expenditure information of the user;
a food sample nutritional information measuring device transmitting means external of the processor, for providing food sample nutritional information of a food sample; and
a biological information measuring device transmitting means external of the processor, for providing biological information of the user.

7. The device of claim 1, wherein the input means further comprises communication means.

8. The device of claim 7, wherein the communications means communicates with a global telecommunications network.

9. The device of claim 7, wherein the communications means includes wireless communication means for communicating with at least one external device.

10. The device of claim 1, wherein the output means further comprises:
means responsive to a procedure for generating activation signals adapted to control a pharmaceutical delivery system carried on the user.

11. The device of claim 1, further comprising a memory.

12. The device of claim 11, wherein the operating program executed by the processor is stored in the memory.

13. The device of claim 11, wherein the output means comprises a display.

14. The device of claim 11, wherein the processor, the memory, the input means and the output means are disposed in a handheld housing.

15. The device of claim 1, wherein the processor, the input means and the output means are disposed in a handheld housing.
